(19) United States
(12) Patent Application Publication
(10) Pub. No.: US 2013/0089568 A1
(43) Pub. Date: Apr. 11, 2013

(54) HAIR GROWTH STIMULATOR BASED ON ECKLONIA CAVA EXTRACT, PREPARATION METHOD THEREOF, AND COSMETIC COMPOSITION CONTAINING THE SAME

(71) Applicants: Se-Kwon KIM, Busan (KR); Jung-Ae KIM, Busan (KR); Byul-Nim AHN, Busan (KR); Soon-Sun BAK, Busan (KR)

(72) Inventors: Se-Kwon KIM, Busan (KR); Jung-Ae KIM, Busan (KR); Byul-Nim AHN, Busan (KR); Soon-Sun BAK, Busan (KR)

(21) Appl. No.: 13/647,601

(22) Filed: Oct. 9, 2012

(30) Foreign Application Priority Data
Oct. 7, 2011 (KR) 10-2011-0102474

(51) Int. Cl. A61K 8/49; A61Q 7/00; C07D 493/04

(52) U.S. Cl. 424/195.17; 549/358

(57) ABSTRACT

The present invention relates to a hair growth stimulator based on an Ecklonia cava extract, a preparation method thereof and a cosmetic composition containing the same. Dioxinodehydroeckol is isolated from the Ecklonia cava extract. The Ecklonia cava extract prepared according to the preparation method and the dioxinodehydroeckol compound have excellent hair growth effects which are significantly higher than the effect of an Ecklonia cava extract prepared according to a conventional method.

### CROSS-REFERENCE TO RELATED APPLICATION(S)

This application claims the benefit under 35 U.S.C. §119(a) of Korean Patent Application No. 10-2011-0102474 filed on Oct. 7, 2011, in the Korean Intellectual Property Office, the entire disclosure of which is incorporated herein by reference for all purposes.

### BACKGROUND OF THE INVENTION

1. Field of the Invention

The present invention relates to a hair growth stimulator based on an Ecklonia cava extract, a preparation method thereof, and a cosmetic composition containing the same.

More specifically, the present invention relates to a purified extract of Ecklonia cava for hair growth, a dioxinodehydroeckol compound isolated therefrom, and a cosmetic composition containing the same, which act on human hair follicles, human follicle dermal papilla cells (HFDPC) and C57BL/6 mice to stimulate hair growth.

2. Description of the Prior Art

Human hairs play very important not only in protecting the skin and scalp, but also in social and sexual communication. Thus, studies on hair growth and hair loss have been continuously conducted, but a mechanism associated therewith has not yet been clearly found.

Hairs are composed of keratin protein, and develop and grow from hair follicles in the dermis. Scalp hair follicles are composed of follicle dermal papilla cells, inner and outer (Inner root sheath cells, and melanocytes, among them, follicle dermal papilla cells are special keratinocytes on which hair follicle development is based. Follicle dermal papilla cells are known to have a close connection with hair growth.

Recently, studies on many regulators involved in hair growth and hair loss mechanisms have been actively conducted, and particularly, it has been reported that various factors associated with a cell cycle of anagen, catagen and telogen are regulated through signaling by their receptors.

It is known that extracellular signal-regulated kinases (ERKs) and a serine/threonine protein kinase (AKT) pathway are involved in the proliferation of scalp follicle dermal papilla cells. In addition, it is known that p53, Bcl-2 and Bax are influenced by the apoptosis of scalp follicle cells and keratinocytes. Recent results revealed factors related to hair growth, and among them, ALP (alkaline phosphatase), VCAN (versican), KGF (keratinocyte growth factor), VEGF (vascular Endothelial Growth Factor), β-catenin, Wnt signal and the like are known to be factors related to hair growth stimulation. As a method for measuring the hair growth effect of a test substance, hair tissue culture is carried out, and in general, mouse mustaches and human scalp hairs are mainly used.

To evaluate the induction of anagen hair growth using an animal model, C57BL/6 mice are mainly used. In C57BL/6 mice, melanocytes that synthesize a pigment are present specifically in the follicles and the synthesis of melanin pigment occurs only in the anagen phase, and thus the skin color of the mice is black in the anagen phase and is pink in the catagen and telogen phases. With respect to the hair cycle of C57BL/6 mice, the skin of the mice enters the telogen phase 6-7 weeks after birth. Based on this characteristic, when the mouse hair in the telogen phase are pulled out and then the change in the mouse skin is observed while the mice are treated with a test substance over several weeks, the effect of the test substance on the induction of the anagen stage can be evaluated.

With respect to the stimulation of hair growth, various technologies have been reported. For example, Korean Patent Application No. 10-2008-0096649 discloses a hair or scalp cosmetic composition for preventing hair loss and stimulating hair growth, which comprises zinc pyrithione, panthenone and salicylic acid as active ingredients. Further, Korean Patent Application No. 10-1992-0702114 discloses a method and formulation for stimulating hair growth and treating skin disease, wherein the method comprises topically applying a composition comprising anol and atenolol to a patient to stimulate hair growth. In addition, Korean Patent Application

No. 10-1991-0016248 discloses a composition for stimulating hair growth, which contains extracts of Cornis fructus, Eclipta prostrate and mulberry fruits.

As described above, a variety of herbal drug for stimulating hair growth have been reported, whereas stimulators derived from natural marine organisms are scarcely known. Korean Patent Application No. 10-2008-18124 discloses a hair growth composition for increasing the antibacterial activity of scalp and stimulating hair growth, which comprises extracts of various seaweeds.

Natural marine organisms have many useful physiologically active compounds, and thus many studies on the development and application of natural marine organisms have recently been conducted. Among natural marine organisms, seaweeds are classified, according to pigment, Phaeophyceae, Rhodophyceae and Chlorophyceae.

It was reported that Ecklonia cava (E. cava), a seaweed which is used in the present invention, lives in the sea around Korea and Japan and has anticoagulant, antioxidant, anticancer and matrix metalloproteinase (MMP) inhibitory activities. In the prior art, it was reported that a purified extract of Ecklonia cava, which is a physiologically active compound isolated from Ecklonia cava, and a dioxinodehydroeckol compound, have inhibitory effects on adipocyte differentiation and melanin formation.

The present inventors have tried to prepare an Ecklonia cava extract, which has improved activity and effects so as to be industrially applicable, by carrying out a series of extraction and purification procedures, rather than simply confirming whether Ecklonia cava has hair growth effects.

Under such circumstances, the present inventors have verified the effects of a purified extract of Ecklonia cava and a dioxinodehydroeckol compound isolated therefrom on the proliferation and anti-apoptosis of human follicle dermal papilla cells, and at the same time, have conducted studies on the mechanism of action of hair growth-related factors and carried out a preclinical test using C57BL/6 mice. As a result, the present inventors have found the use of a purified extract of Ecklonia cava and a dioxinodehydroeckol compound as a hair growth stimulator, thereby completing the present invention.

### SUMMARY OF THE INVENTION

As described above, it is an object of the present invention to provide a composition or compound which is based on a natural material and has excellent effects on the stimulation of hair growth.

Another object of the present invention is to prepare an Ecklonia cava extract, which has improved activity and effects so as to be industrially applicable, by properly purifying Ecklonia cava, rather than simply confirming whether Ecklonia cava has hair growth effects.

To achieve the above objects, the present invention provides a hair growth stimulator based on an Ecklonia cava extract.

Herein, the Ecklonia cava extract is preferably dioxinodehydroeckol.

The present invention also provides a method for preparing a hair growth stimulator, the method comprising the steps of:

powdering Ecklonia cava;

adding methanol to the powder Ecklonia cava to obtain an Ecklonia cava solution, extracting the Ecklonia cava solution by stirring, and filtering the extract;

concentrating the filtered extract;

homogenizing the concentrate by adding distilled water thereto;

adding hexane to the homogenized concentrate, stirring the hexane-added concentrate, and removing a hexane layer from the stirred concentrate;

adding dichloromethane to a water layer remaining after removal of the hexane layer, stirring the dichloromethane-added water layer, and removing a dichloromethane layer from the stirred water layer; and

adding ethyl acetate to the water layer remaining after removal of the dichloromethane layer, stirring the ethyl acetate-added water layer, and collecting and concentrating an ethyl acetate layer therefrom.

The present invention also provides a hair growth stimulator based on an Ecklonia cava extract prepared by the above method.

The present invention also provides a cosmetic composition containing the above hair growth stimulator.

### BRIEF DESCRIPTION OF THE DRAWINGS

FIG. is a set of graphs showing the effects of a methanol extract of Ecklonia cava (1a), a purified extract of Ecklonia cava (1b) and a dioxinodehydroeckol compound (1c) on the proliferation of human follicle dermal papilla cells and shows a comparison between test results for the effects of a hexane extract (H), a dichloromethane extract (DC) and a purified extract of Ecklonia cava (EA) on the proliferation of human follicle dermal papilla cells.

FIG. 2 shows the effects of a purified extract of Ecklonia cava (2a) and a dioxinodehydroeckol compound (2b) on the proliferation of human follicle dermal papilla cells and on hair growth-related factors.

FIG. 3 is a set of graphs showing the hair growth effects of a purified extract of Ecklonia cava (3a) and a dioxinodehydroeckol compound (3b) in the culture of human hair tissue.

FIG. 4 is a set of photographs showing test results for the effects of a purified extract of Ecklonia cava on the induction of the anagen phase.

### DETAILED DESCRIPTION OF THE INVENTION

Hereinafter, the present invention will be described in detail with reference to specific test examples.

1. Preparation of Purified Extract of Ecklonia cava and Dioxinodehydroeckol Compound

A. Preparation of Purified Extract of Ecklonia cava

(1) Dried Ecklonia cava was powdered, and the powder was extracted three times by stirring with a 3-fold weight of methanol, and the extract was filtered.

(2) The filtrate was concentrated in a rotary vacuum evaporator to a purity of 99.9%, thereby a methanol extract (hereinafter referred to as the “methanol extract of Ecklonia cava”).

(3) adding a 10-fold volume of distilled water to the methanol extract of Ecklonia cava and sufficiently homogenizing the methanol extract of Ecklonia cava using an ultrasonic washer.

(4) The same amount of hexane as that of the added distilled water was added to the homogenized extract, which was then stirred, and a hexane layer was removed therefrom (repeated three times).

(5) The same amount of dichloromethane was added to a water layer remaining after removal of the hexane layer, and then the water layer was stirred, after which a dichloromethane layer was removed therefrom (repeated three times).

(6) The same amount of ethyl acetate was added to the water layer remaining after removal of the dichloromethane layer, and then the water layer was stirred, after which an ethyl acetate layer was collected therefrom and concentrated in a rotary vacuum evaporator, thereby preparing an Ecklonia cava extract (hereinafter referred to as the “purified extract of Ecklonia cava”) which is the ethyl acetate layer.

B. Dioxinodehydroeckol Compound

The ethyl acetate layer obtained by the above method was subjected to column chromatography and Sephadex LH-20 column chromatography to isolate an active compound fraction. The isolated compound was dioxinodehydroeckol having the following structural formula:

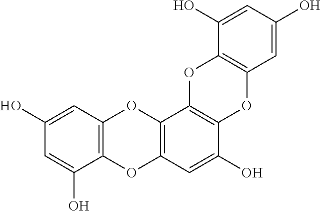 [SMILES: Oc1cc(O)c2oc3cc(O)c4oc5cc(O)cc(O)c5oc4c3oc2c1]

<Structure of Dioxinodehydroeckol Compound Isolated from Ecklonia cava>

Various active substances isolated and purified from Ecklonia cava in the procedure of the present invention were tested using hair-related cells in an in vitro system, and as a result, it was found that a 7′-fluoroeckol compound and a dioxinodehydroeckol compound specifically influence hair-related cells. The 7′-fluoroeckol compound is disclosed in Korean Patent Application No. 10-2011-0000141. The present invention aims to find a dioxinodehydroeckol compound and an Ecklonia cava extract using an extraction method capable of improving hair growth effects.

Accordingly, the present inventors carried out the structural analysis of the active substance purified Ecklonia cava extract and the isolated dioxinodehydroeckol compound using a verified method.

2. Test Method

A. Cell Culture

Human follicle dermal papilla cells were purchased from Promo Cell (Heidelberg, Germany). The cells were cultured in a ready-to-use HFDPC medium composed of a basal medium, FCS (fetal calf serum) and a supplement kit Promo Cell (Heidelberg, Germany) containing basal FGF and insulin. For subculture, a kit containing HBSS (HEPES buffered saline), trypsin/EDTA solution and neutralizing solution (Promo Cell, Heidelberg, Germany) was used. Human follicle dermal papilla cells at passages 4 to 6 were used in the test and cultured in a T-25 flask containing 5 ml of medium under the conditions of 37° C. and 5% CO₂.

B. Cell Viability

The cell viability of human follicle dermal papilla cells were measured using a 3-(4,5-dimethylthiazole-2-yl)-2,5-diphenyl-tetrazolium bromide [MTT] assay.

Specifically, 100 μL of cells were dispensed into each well of 96-well plates at a concentration of 1×10⁶ cells/ml and cultured for 24 hours. A sample was prepared in a medium free of FBS and 1% penicillin/streptomycin at various concentrations, and the cells was treated with the sample and incubated for 24 hours. The cells were treated with an MTT solution (5 mg/mL) for 3 hours, and then the MTT was reduced and the medium was carefully removed such that the produced formazan was not removed. Then, 100 μL of dimethyl sulfoxide (DMSO) was dispensed into each well, and after 30 minutes, the absorbance at 570 nm was measured.

C. Protein Expression Test

In order to examine the mechanisms of action of the purified extract of Ecklonia cava and the dioxinodehydroeckol compound on the proliferation of human follicle dermal papilla cells, Western blotting was carried out according to standard protocol.

Specifically, the cells were seeded into a 6-well medium together with a serum-free medium and cultured for 24 hours (1.0×10⁶ cells/well). Then, the medium was replaced with a fresh medium, and the cells were treated with various concentrations of a sample for 1-24 hours. The cells were lysed in RIPA cell lysis buffer at 4° C. for 10 minutes. 10 μg of the cell lysate was separated by 10% SDS-polyacrylamide gel electrophoresis, transferred to a polyvinylidene fluoride membrane, blocked with 5% non-fat milk, and hybridized with primary antibody (diluted at 1:10000). The cells were cultured with horseradish-peroxidase-conjugated secondary antibody at room temperature, and then immune-reactive protein was detected using an enhanced chemiluminescence (ECL) analysis kit according to the manufacturer's instruction. The Western blot bands were visualized using LAS3000® Luminescent image analyzer (Fujifilm Life Science, Tokyo, Japan).

D. mRNA Expression

In order to examine the mechanisms of action of the purified extract of Ecklonia cava and the dioxinodehydroeckol compound on the proliferation of human follicle dermal papilla cells, reverse transcription polymerase chain reaction (RT-PCR) was performed according to a standard protocol. Specifically, the cells were seeded into a 6-well plate together with a serum-free medium and cultured for 24 hours (1.0×10⁶ cells/well). Then, the medium was replaced with a fresh medium, and the cells were treated with various concentrations of a test material for 24 hours and then lysed with TRIzol® (Invitrogen).

E. Evaluation of Hair Growth by Culture of Human Hair Tissue

Human hair tissue provided from Immunobiology Laboratory, Kyungpook National University, was used in the test and cultured in a CO₂ incubator at 37° C. with Williams E media (Sigma, USA) containing 2 mM L-glutamin, 100 U/ml streptomycin and 10 ng/ml hydrocortisone. The tissue was cultured with various concentrations of the test material for 6 days, and the length of the hair tissue was measured at days 3 and 6.

F. Evaluation of Effect of Hair Growth Stimulator Using Animal Model

8-week-old female C57BL/6 mice were purchased from Orient Bio Co. (Gyeonggi-do, Korea) and used in the test. 6 or more mice were used per group, and the back of each mouse was shaved with an electric shaver. The test material was applied so the shaved portion once a day, five times a week for 7 weeks. The mice were observed visually while they were photographed at 1-week intervals, whereby the effect of the test material on the induction of the anagen phase was measured.

G. Statistical Analysis

The data obtained in the above test were expressed in mean±SEM. The data of each group were subjected to one-way ANOVA by Duncan's multiple range test using the statistical software package SAS v9.1 (SAS Institute Inc., North Carolina, USA). The difference was considered significant at p<0.05.

3. Test Procedures and Results

A. Cell Viability

(1) Ecklonia cava was extracted with a solvent, and a test on the proliferation of human follicle dermal papilla cells having a close connection with hair growth was carried out using the extract.

When the cells were treated with the methanol extract of section 1-A-(2), the cell viability was increased at several concentrations (0.1, 1 and 1000 μg/ml) compared to the control group, but was similar to one obtained by Minoxidil that is the main component of a hair growth stimulator which is currently typically used (FIG. 1a)

Thus, the present inventors tried to increase this activity of the methanol extract of Ecklonia cava through a specific purification procedure.

(2) When human follicle dermal papilla cells were treated with the purified extract of Ecklonia cava of section 1-(A)-(6) (FIG. 1b), the viability of the human follicle dermal papilla cells was significantly increased in the concentration range of 0.001-0.05 μg/ml. Particularly, the cell viability was 129.8% at a concentration of 0.05 μg/ml, which was significantly higher than when the cells were treated with 1 μM of Minoxidil. In the concentration range of 0.1-10 μg/ml, the purified extract of Ecklonia cava showed high cell viability compared to the group not treated with the purified extract of Ecklonia cava, but the cell viability was lower than that at a concentration of 0.05 μg/ml. In comparison with Minoxidil known to have a hair growth effect, the cell, viability when treated with 1 μM of Minoxidil (general concentration for comparison) was 118.8%, which was lower than that obtained when treated with the purified extract of Ecklonia cava.

It is known that human cells have the respective specific properties, and among them, cell viability (proliferation rate) differs between cells. It is generally known that the division cycle of normal cells is 24 hours, whereas the division cycle of cancer cells, particularly stomach cancer cells, is shorter than 12 hours. It is known that human follicle dermal papilla cells have a very slow proliferation rate and a division cycle of about 48 hours, unlike other general cells. Thus, the difference in proliferation rate (%), which appeared when the cells were treated with the test material for 4 days (96 hours) in the test method used in the present invention, can be considered significant.

Also, the evaluation of the effect of a test material is generally performed through three steps. The first step is the evaluation of the effect using an in vitro system, the second step is the evaluation of the effect using an animal model, and the third step is clinical evaluation which is applied to the human body. Further, when a material effective in the in vitro system is found, the verification of the effect thereof is performed in an animal model at concentrations 100 times and 1,000 times the maximum treatment concentration of the sample. The clinical evaluation is also performed at concentrations similar to or about 10 times higher than the above concentrations. Thus, if an animal test is performed at the maximum concentration confirmed to be effective in a test for an increase in the proliferation rate of human follicle dermal papilla cells, which was used in the present invention, a test can be performed at the following concentration.

Specifically, in the case of the methanol extract of Ecklonia cava, an animal test should be performed at a minimum concentration of 100 mg/ml, because the maximum concentration confirmed in the cell test is 1000 μg/ml (=1 mg/ml). Even if the results of the verification of the test material indicate that the test material has an effect, the test material is difficult to apply industrially, because the concentration thereof is excessively high. For this reason, in the present invention, a purified extract of Ecklonia cava was prepared through a series of purification procedures such that it can be industrially used. The maximum concentration of the purified extract of Ecklonia cava prepared in the present invention was confirmed to be 0.05 μg/ml in the cell test, and thus the purified extract of Ecklonia cava was used at a minimum concentration of 5 μg/ml in an animal test. In addition, the maximum concentrations of the methanol extract of Ecklonia cava and the purified extract of Ecklonia cava are 1000 μg/ml and 0.05 μg/ml, respectively, which are numerically significantly different from each other. As described above, the methanol extract of Ecklonia cava is industrially invaluable, because the amount of Ecklonia cava used is excessively large.

(3) When human follicle dermal papilla cells were treated with a dioxinodehydroeckol compound isolated from Ecklonia cava (FIG. 1c), the viability of the human follicle dermal papilla cells was significantly increased in the concentration range of 0.001-5 μM compared to that of a control group not treated with the compound. The cell viability concentration-dependently increased in the concentration range of 0.001-0.1 μM, and the highest cell viability was 142.1% at a concentration of 0.1 μM. When the cells were treated with 1 μM of minoidil, the cell viability was 121%, which was lower than when the cells were treated with the dioxinodehydroeckol compound.

(4) Meanwhile, as shown in FIG. 1d, a cell proliferation test for human follicle dermal papilla cells was performed using (1) the methanol extract of Ecklonia cava (M) of section 1-A-(2), (2) the purified extract of Ecklonia cava (EA) of section 1-A-(6), (3) a hexane extract of Ecklonia cava (H) obtained by collecting the hexane layer of section 1-A-(4) and concentrating the collected hexane layer in a rotary vacuum evaporator, and (4) a dichloromethane extract of Ecklonia cava (DC) obtained by collecting the dichloromethane layer of section 1-A-(5) and concentrating the collected dichloromethane layer in a rotary vacuum evaporator. As a result, it was found that the purified extract of Ecklonia cava (EA) among the fractions of Ecklonia cava had a significantly increased effect on cell proliferation compared to other fractions.

B. Protein and mRNA Expression

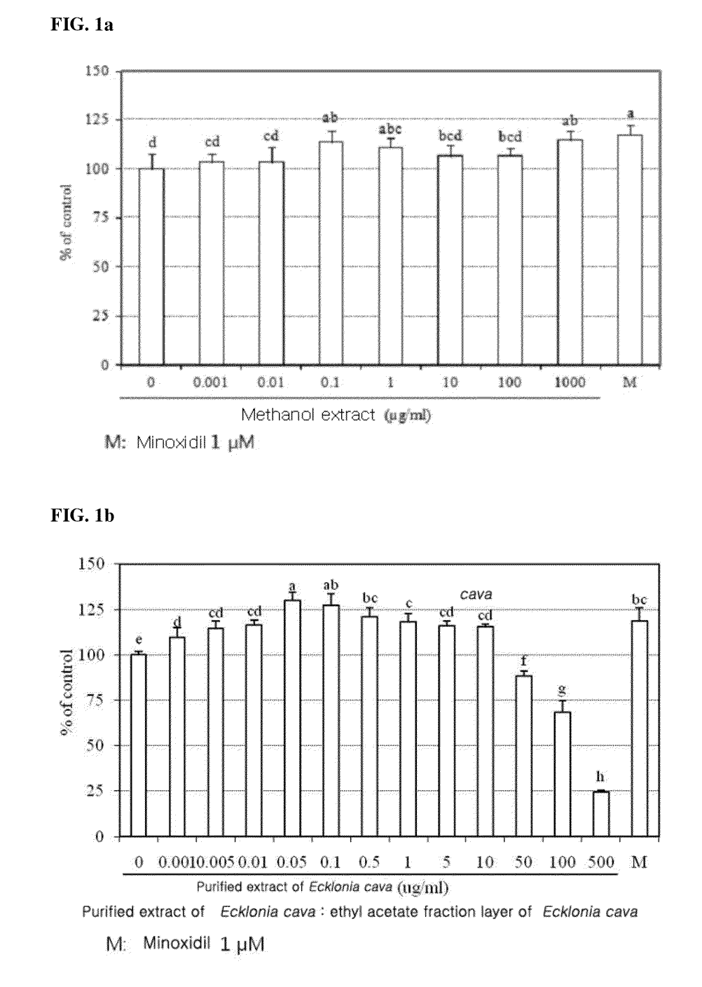

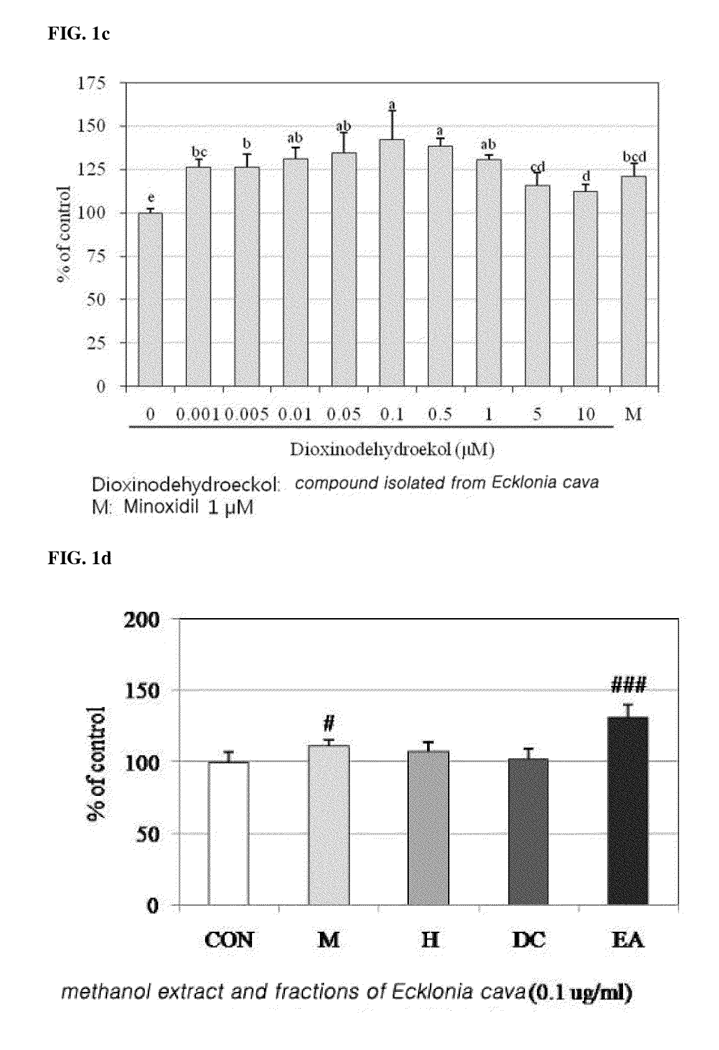

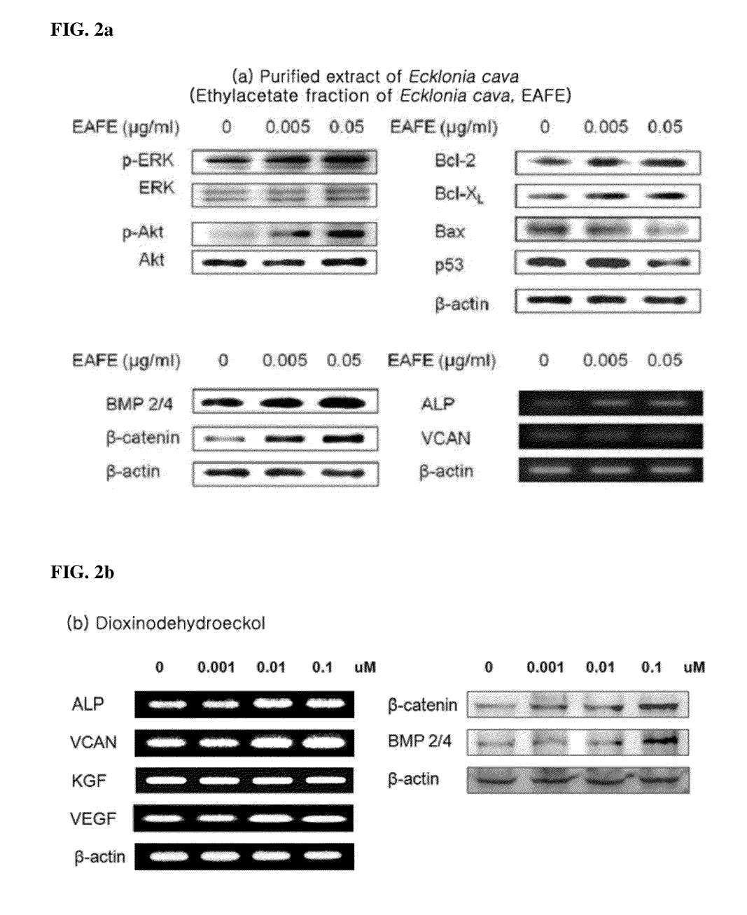

(1) In order to examine protein expression by Western blot analysis, human follicle dermal papilla cells were treated with the purified extract of Ecklonia cava for 1-24 hours and subjected to Western blot analysis. The results of the Western blot analysis are shown in FIG. 2a.

For a test for ERK and AKT pathways which are involved in cell proliferation, human follicle dermal papilla cells were treated with the purified extract of Ecklonia cava for 1 hour. Also, in order to observe changes in apoptosis-related factors, including Bcl-2 and Bcl-X_{L}, which are anti-apoptosis factors, Bax and p53, which are pro-apoptosis factors, and ALP, VCAN, β-catenin and BMP 2/4, which are hair growth-related factors, human follicle dermal papilla cells were treated with the purified extract of Ecklonia cava for 24 hours, and then changes in the proteins were observed.

The expression levels of phosphorylated ERK (p-ERK) protein in the human follicle dermal papilla cells treated with the purified extract of Ecklonia cava at concentrations of 0.005 and 0.05 μg/ml were increased by 1.3 times and 1.2 times, respectively, compared to that in the control group, based on the total AKT level. The expression levels of phosphorylated AKT (p-AKT) protein in the human follicle dermal papilla cells treated with the purified extract of Ecklonia cava at the same concentrations were increased by 2.8 times and 2.5 times, respectively, based on the total AKT level. The expression levels of Bcl-2 and Bcl-X_{L} proteins in the human follicle dermal papilla cells treated with the purified extract of Ecklonia cava at a concentration of 0.05 μg/ml were increased by 1.6 times and 1.9 times, respectively, and these increases in the protein expression levels were dependent on the concentration of the purified extract of Ecklonia cava. The expression levels of Bax and p53 proteins in the human follicle dermal papilla cells treated with the purified extract of Ecklonia cava at the same concentration were decreased by 0.5 times and 0.8 times, respectively (p<0.05).

From the above results, it can be seen that the purified extract of Ecklonia cava increases the expressions of phosphorylated ERK and AKT (cell proliferation factors) in human follicle dermal papilla cells known to have a high connection with hair formation and growth, thereby promoting the proliferation of the human follicle dermal papilla cells. Moreover, it can be seen that the purified extract of Ecklonia cava regulates the expressions of the anti-apoptotic factors Bcl-2 and Bcl-X_{L} and the pro-apoptotic factors Bax and p53 to induce the proliferation of human follicle dermal papilla cells. Apoptosis means natural cell death and is determined by the interaction of anti-apoptosis and pro-apoptosis proteins, and natural cell death occurs through this series of procedures. It is believed that the purified extract of Ecklonia cava inhibits the natural death of human follicle dermal papilla cells such that the cells remain in the anagen phase.

(2) In order to examine the changes in hair growth-related factors caused by the purified extract of Ecklonia cava, changes in ALP, VCAN, β-catenin and BMP 2/4 were observed. Specifically, human follicle dermal papilla cells were treated with the purified extract of Ecklonia cava for 24 hours, and then the expression levels of proteins or mRNAs in the cells were observed. The expression levels of BMP2/4 and β-catenin proteins in the cells were significantly increased by the purified extract of Ecklonia cava in a manner dependent on the concentration of the purified extract of Ecklonia cava. In addition, it was shown that the expression levels of mRNAs of ALP and VCAN increased as the concentration of the purified extract of Ecklonia cava increased.

Recent studies revealed that ALP, VCAN, β-catenin and BMP 2/4 are factors that promote hair formation and growth. Thus, it can be seen that the purified extract of Ecklonia cava regulates factors that promote hair formation and growth in human follicle dermal papilla cells, thereby inducing the expression of the factors.

(3) In order to examine the changes in hair growth-related factors caused by dioxinodehydroeckol, changes in mRNAs and proteins were analyzed by RT-PCR and Western blot analysis. The results of the analysis are shown in FIG. 2b.

Specifically, in order to observe changes in ALP, VCAN, KGF, VEGF, β-catenin and BMP 2/4, which are hair growth-related factors, human follicle dermal papilla cells were treated with dioxinodehydroeckol for 24 hours. When the human follicle dermal papilla cells were treated with 0.1 μM of dioxinodehydroeckol, the expression levels of BMP2/4 and β-catenin proteins in the cells were increased by 1.8 times and 2.0 times, respectively, compared to that in a control group not treated with the sample (p<0.05). Moreover, the expression levels of ALP, VCAN and VEGF mRNAs at the same concentration of dioxinodehydroeckol as above were increased by 1.5 times, 1.8 times and 1.3 times, respectively (p<0.05).

It is known that ALP, VCAN, KGF, VEGF, β-catenin and BMP 2/4 are factors that promote hair formation and growth. Thus, it can be seen that dioxinodehydroeckol regulates factors that promote hair formation and growth in human follicle dermal papilla cells, thereby inducing the expression of the factors.

C. Evaluation of Hair Growth by Culture of Human Hair Tissue

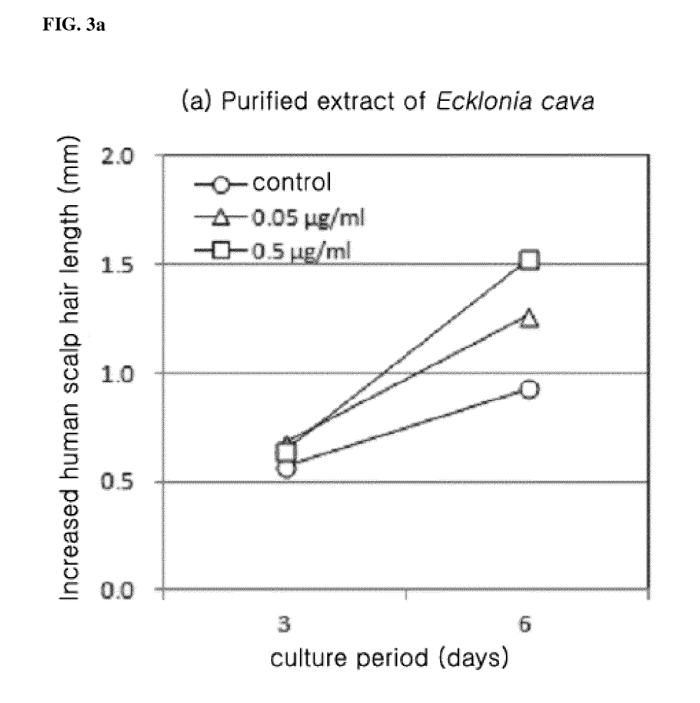

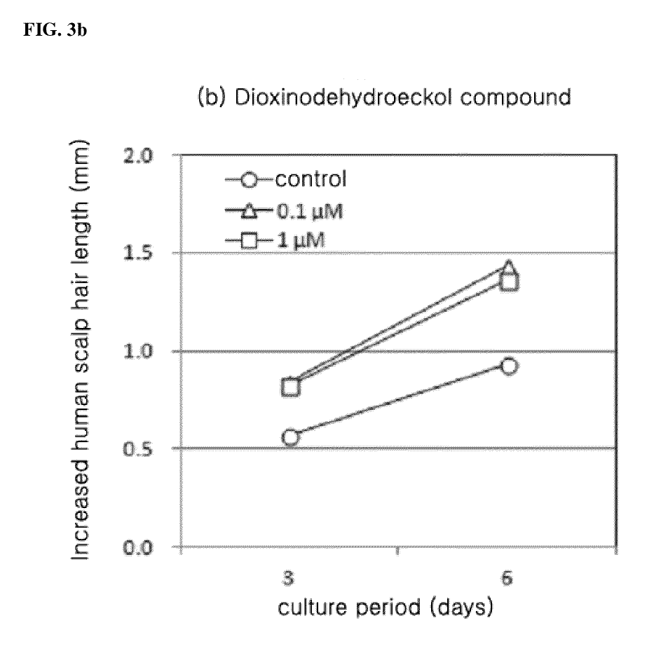

The culture of human hair tissue was used to examine the hair growth effects of the purified extract of Ecklonia cava and the dioxinodehydroeckol compound (FIGS. 3a and 3b).

Human hair tissue was treated with various concentrations of each of the test materials, and then the increased hair length was measured at days 3 and 6. A control group not treated with the test material showed 0.57 mm at day 3 and 0.93 mm at day 6, whereas the group treated with 0.5 μg/ml of the purified extract of Ecklonia cava showed increases in human hair length to 0.68 mm at day 3 and 1.26 mm at day 6. Also, the group treated with 0.1 μM of the dioxinodehydroeckol compound showed 0.84 mm at day 3 and 1.43 mm at day 6, which were longer than those in the control group not treated with the test material.

D. Evaluation of Hair Growth Stimulator Using Animal Model

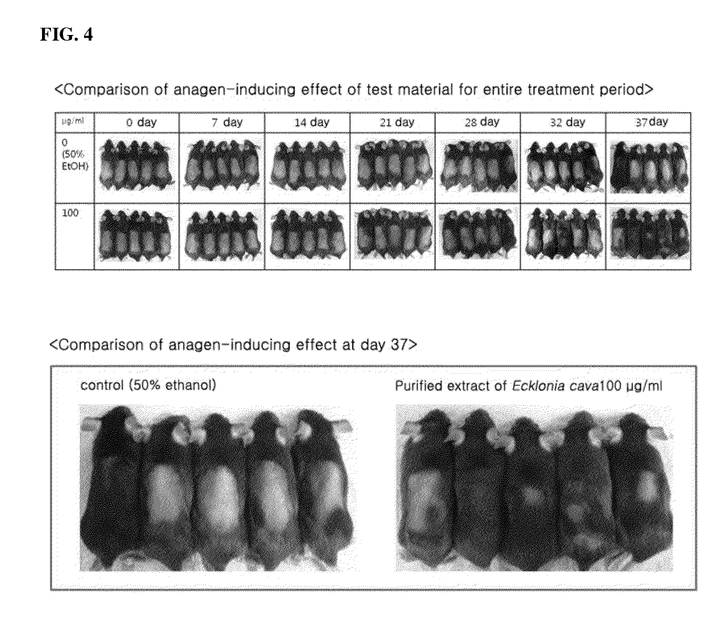

Using 8-week-old female C57BL/6 mice whose skin state entered the telogen phase of the hair cycle such that the skin surface was white, the effect of the purified extract of Ecklonia cava on the induction of the anagen phase was evaluated (FIG. 4).

In the control group (treated with 50% ethanol) not treated with the test material, the shaved skin area of one mouse changed to a black color at day 28 (characteristic of the anagen phase of C57BL/6 mice), but other four mice showed this phenomenon at day 37. On the other hand, in the group treated with the purified extract of Ecklonia cava at a concentration of 100 μg/ml, the shaved skin area started to change to a black color from day 21. In this group, it could be observed that the anagen phase was already initiated in all the five mice at day 32, although the degree of the change did differ between the mice. At day 37 (the final day of the test), in the control group not treated with the test material, the characteristic of the anagen phase was observed, whereas in the mice treated with the purified extract of Ecklonia cava, it was observed that new hair grew at the shaved area, although the degree of the hair growth did differ between the mice.

E. Conclusion

It was shown that the purified extract of Ecklonia cava containing the dioxinodehydroeckol compound increased the viability of human follicle dermal papilla cells compared to the methanol extract of Ecklonia cava. This is thought to be because the purified extract of Ecklonia cava containing the dioxinodehydroeckol compound acts in human hair cells in a manner similar to growth factors to stimulate the growth of human hair tissue and functions to induce the anagen phase in mice in the telogen phase.

Thus, it is thought that the purified extract of Ecklonia cava and the dioxinodehydroeckol compound, which have the above-described characteristics, are industrially highly valuable.

As described above, the extract of Ecklonia cava prepared according to the present invention and the dioxinodehydroeckol isolated therefrom have excellent hair growth effects. The test results show that these effects are significantly higher than an Ecklonia cava extract prepared according to a conventional method.

Thus, it is thought that the hair growth stimulator of the present invention is industrially highly valuable.

What is claimed is:

1. A hair growth stimulator based on an Ecklonia cava extract.

2. The hair growth stimulator of claim 1, wherein the Ecklonia cava extract is dioxinodehydroeckol.

3. A method for preparing a hair growth stimulator, the method comprising the steps of:
powdering Ecklonia cava;
adding methanol to the powder Ecklonia cava to obtain an Ecklonia cava solution, extracting the Ecklonia cava solution by stirring, and filtering the extract;
concentrating the filtered extract;
homogenizing the concentrate by adding distilled water thereto;
adding hexane to the homogenized concentrate, stirring the hexane-added concentrate, and removing a hexane layer from the stirred concentrate;
adding dichloromethane to a water layer remaining after removal of the hexane layer, stirring the dichloromethane-added water layer, and removing a dichloromethane layer from the stirred water layer; and
adding ethyl acetate to the water layer remaining after removal of the dichloromethane layer, stirring the ethyl acetate-added water layer, and collecting and concentrating an ethyl acetate layer therefrom.

4. A hair growth stimulator based on an Ecklonia cava extract prepared by the method of claim 3.

5. A cosmetic composition containing the hair growth stimulant of any one of claims 1, 2 and 4.
